(19) United States
(12) Patent Application Publication
(10) Pub. No.: US 2008/0077163 A1
(43) Pub. Date: Mar. 27, 2008

(54) Anti-Fistula Strip

(75) Inventor: David Jean-Marie Nocca, Montpellier (FR)

Correspondence Address: WOLF GREENFIELD & SACKS, P.C., 600 ATLANTIC AVENUE, BOSTON, MA, 02210-2206, US

(73) Assignee: Davol, Inc., Cranston, RI (US)

(21) Appl. No.: 11/597,485

(22) PCT Filed: May 17, 2005
(86) PCT No.: PCT/FR05/01230

§ 371 Date: Apr. 9, 2007

(30) Foreign Application Priority Data
May 25, 2004 (FR) 04 05617

(51) Int. Cl. A61B 17/11

(52) U.S. Cl. 606/154

(57) ABSTRACT

The inventive strip is made of a resorbable material and colonisable by fibroblasts comprises a closure for connecting two ends, thereby securing an anastomosis.

Use ofa digestive or biliary anastomosis, whether done by hand or with the help of a mechanical clamp, is very common in abdominal surgery (especially in the treatment of intestinal blockage).

The most frequent (and possibly life-threatening) complication is the occurrence of an anastomotic fistula resulting from lack of tightness of the anastomosis. This will cause a case of peritonitis due to the intraluminal contents getting into the peritoneal cavity; any perforation of the digestive tube can also cause this same complication.

The invention mitigates this danger by a flexible, non-elastic strip, all in one piece, made of resorbable material, having a longitudinal axis, containing a closure allowing one to join its two end pieces into a ring, one of the end pieces being wider than the other, which is narrow, characterized in that the widened piece is pierced by at least one pair of suture openings, the openings of one pair being arranged symmetrically with respect to the longitudinal axis of the strip.

By flexible is meant that the strip can be curved into a ring without breaking.

Thanks to the choice of a r esorbable material, the implant will be colonized by fibroblasts and revascularized. Experimental trials have shown a good toughness for a thickness comparable to knitted synthetic canvas. Using the means of joining the two ends of the band, one can adapt the diameter of the ring fonmed by curving the strip while maintaining its shape, so as to ensure good tightness.

Preferably. the means are comprised of conformations of the strip itself and even better, they are comprised solely of conformations of the strip itself. Thus, the means can be comprised of a slit near one of the ends and by a tongue joined to the other end, which can be inserted into the slit when the strip is curved into a ring.

Preferably, one end part of the strip has a greater width than the slit, so as to ensure tightness with respect to the slit. This end part of greater width advantageously has, in its four corners, openings for fixation to the viscera by a thread passing through them.

The slit, in particular, has a width of 1 to 2 mm, corresponding to the thickness of the strip, and a length just greater than the width of the main piece of the strip.

To ensure good tightness, it is preferable for each opening to have a dimension between 1 and 3 mm and for the openings of one pair to be punctured at a distance from the edge of the widened piece between 2 mm and 5 mm. In this way, one ensures both a good suturing and no deformation of the strip. It is preferable that the openings of one pair be spaced apart from each other by a distance greater than the width of the narrow piece so as to support the strip properly and thereby avoid any lack of tightness.

According to one especially favored embodiment, the widened piece has an oval shape, which still ensures good tightness, but is less blunt.

Preferably, a visual reference extends transversely on the narrow piece, making it possible to define a minimum diameter of the strip after being positioned around the viscera. The surgeon is thus certain not to tighten the tubular organ of the body too much.

According to a preferred embodiment, two openings of a pair are aligned with the longitudinal direction of the slit, and only these two openings are provided for one suture. Thus, the tightening of the strip is well balanced and tightness is better ensured.

As resorbable products which can be used, one can mention:
collagen of human or animal origin, especially porcine,
intestinal submucosa of animal origin,
vesical submucosa of animal origin, and
bovine pericardium.

One can mention, as type of anastomosis and minimum size to prevent stenosis:
the colorectal and ileorectal anastomosis: minimum size 15 mm internal diameter,
the choledochojejunal anastomosis: minimum size 6 mm internal diameter,
the pancreaticjejunal anastomosis: minimum size variable, depending on the size of the pancreatic stump,
the esogastric anastomosis: minimum size 12 mm internal diameter,
the gastrojejunal anastomosis: minimum size 12 mm internal diameter.

Finally, the invention deals with a method of digestive or biliary anastomosis or treatment for perforations of the digestive tube, in which one encircles a tubular organ of the body with a strip according to the invention and sutures the strip to a viscera of the body by the suture openings of the strip.

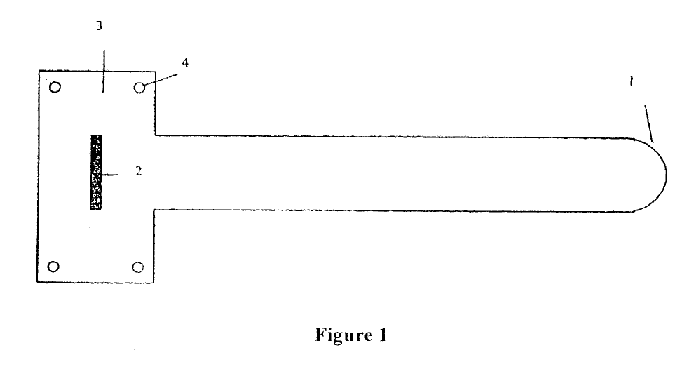

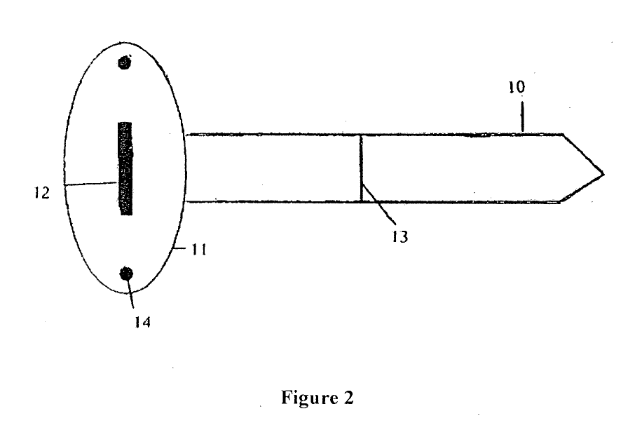

FIGS. 1 and 2 of the enclosed drawing illustrate the invention.

FIG. 1 is a plan view of a pig collagen strip, 12 cm in length and 3 cm in width. At one ofthe ends there is a tongue 1, rounded with a diameter of 3 cm, while at the other end there is a slit 2 of 3, 2 [3.2?] cm length, extending perpendicularly to the direction running from one end of the strip to the other, such that after the strip is curved into a ring one can insert the tongue 1 into the slit 2, thereby closing the ring in a tight manner, and having the possibility of adjusting the diameter of the ring.

The slit 2 is made in a widened part 3, being 3 cm in the longitudinal direction of the strip and 7 cm in the direction perpendicular to the largest extension of the strip. At the four corners of the part 3 there are four punctured openings 4 for fixation to the viscera.

FIG. 2 is a plan view of a preferred strip according to the invention, made of pork collagen, 12 cm in length, not counting the pointed part of the tongue, and having a main part 10 with width of 1.5 cm and a widened part 11 in the shape of an ellipse, whose major diameter is 6 cm and whose minor diameter is 3 cm. In this widened part 11, there is made a slit 12, slightly wider than the piece 10, so that the latter can be inserted therein to form a ring. At 5.5 cm from the slit 12, a line 13 is drawn on the ring, allowing the practitioner to define a minimum diameter for the ring that must not be exceeded, to avoid tightening the bodily organ too much. On either side of the slit 12 and aligned with the major diameter of the ellipse, there are only two suture openings 14 in symmetrical fashion, having a diameter of 2 mm. The openings 14 are circular.

The center of the circle of a suture opening is 5 mm away from the closest vertex of the major axis of the ellipse.

1. A flexible, non-elastic strip made of resorbable material and having longitudinal axis, the strip comprising a closure adapted to join two end pieces of the strip into a ring, one of the end pieces being wider than the other to define a widened piece and a narrow piece, the widened piece having at least one pair of suture openings, the openings of one pair being arranged symmetrically with respect to the longitudinal axis of the strip.

2. The strip as claimed in claim 1, wherein each opening has a dimension in a range between approximately 1 mm and approximately 3 mm.

3. The strip as claimed in claim 1, wherein the widened piece has an oval shape.

4. The strip as claimed in claim 3, wherein each opening is located at a distance from an edge of the widened piece, the distance being between approximately 2 mm and approximately 5 mm.

5. The strip as claimed in claim 1, wherein the openings are spaced apart from each other by a distance that is greater than a width of the narrow piece.

6. The strip as claimed in claim 1, further comprising a visual reference extending transversely across the narrow piece.

7. The strip as claimed in claim 6, wherein the narrow piece has a free end that is opposite the widened piece, wherein the visual reference is located closer to the free end of the narrow piece than to the widened piece.

8. The strip as claimed in claim 1, wherein the closure comprises:
a slit having a longitudinal direction that is perpendicular to the longitudinal axis of the strip, the slit being formed in the widened piece, and
a tongue-like formation at a free end of the narrow pieces the tongue-like formation adapted to be inserted into the slit when the strip is curved into a ring.

9. The strip as claimed in claim 8, wherein the two openings of a pair of openings are aligned along the longitudinal direction of the slit.

10. The strip as claimed in claim 1, wherein the resorbable material comprises collagen.

11. A method for digestive or biliary anastomosis or for treatment of perforations of the digestive tube, comprising encircling a tubular organ with the strip according to claim 1 and suturing the strip through the suture openings to a viscera.

12. The method as claimed in claim 11, further comprising inserting the narrow piece through a slit formed in the widened piece.

13. A flexible, non-elastic strip having longitudinal axis, the strip comprising:
a first portion disposed at one end of the strip;
a second portion joined to the first portion and disposed at an opposite end of the strip, the second portion having at least one pair of suture openings adapted to secure the strip to bodily tissue; and
a closure adapted to join the first and second portions together into a ring shape;
wherein the strip is formed of a resorbable material.

14. The strip as claimed in claim 13, wherein the second portion is wider than the first portion.

15. The strip as claimed in claim 13, wherein the suture openings are arranged symmetrically with respect to the longitudinal axis of the strip.

16. The strip as claimed in claim 13, wherein each opening has a dimension in a range between approximately 1 mm and approximately 3 mm.

17. The strip as claimed in claim 13, wherein the second portion has an oval shape.

18. The strip as claimed in claim 17, wherein each opening is located at a distance from an edge of the second portion, the distance being between approximately 2 mm and approximately 5 mm.

19. The strip as claimed in claim 14, wherein the opening are spaced apart from each other by a distance that is greater than a width of the first portion.

20. The strip as claimed in claim 13, further comprising a visual reference extending transversely across the first portion.

21. The strip as claimed in claim 20, wherein the first portion has a free end that is opposite the second portion, wherein the visual reference is located closer to the free end of the first portion than to the second portion.

22. The strip as claimed in claim 13, wherein the closure comprises:
a slit having a longitudinal direction that is perpendicular to the longitudinal axis of the strip, the slit being formed in the second portion, and
a tongue-like formation at a free end of the first portion, the tongue-like formation adapted to be inserted into the slit when the strip is curved into a ring.

23. The strip as claimed in claim 22, wherein the two openings of a pair of openings are aligned along the longitudinal direction of the slit.

24. The strip as claimed in claim 13, wherein the resorbable material comprises collagen.

25. A method for digestive or biliary anastomosis or for treatment of perforations of the digestive tube, the method comprising:
providing a flexible, resorbable, non-elastic strip having longitudinal axis, the strip having a first portion disposed at one end of the strip a second portion joined to the first portion and disposed at an opposite end of the strip, the second portion having at least one pair of suture openings adapted to secure the strip to bodily tissue, and a closure;
encircling a tubular organ with the strip;
using the closure, closing the strip into a ring; and
suturing the strip through the suture openings to bodily tissue.

26. The method as claimed in claim 25, wherein providing the strip with a closure comprises providing the strip having a slit with a longitudinal direction that is perpendicular to the longitudinal axis of the strip, the slit being formed in the second portion, and a tongue-like formation at a free end of the first portion; and wherein closing the strip comprises inserting the tongue-like formation through the slit.
